SEQUENCE LISTING (4 sequences)

SEQ ID NO: 1 - protein, 11 aa, synthetic construct
  REGION 1..11: note = Derived peptide of CD133 aminoacids 40-50
NYETQDSHKAG

SEQ ID NO: 2 - protein, 12 aa, synthetic construct
  REGION 1..12: note = Derived peptide of CD133 aminoacids 559-570
VYSDCKKNRGTY

SEQ ID NO: 3 - protein, 11 aa, synthetic construct
  REGION 1..11: note = Derived peptide of CD133 aminoacids 589-599
EHTGSISSELE

SEQ ID NO: 4 - protein, 10 aa, synthetic construct
  REGION 1..10: note = Derived peptide of CD133 aminoacids 634-643
YLAQTGKSPA

(19) United States
(12) Patent Application Publication
(10) Pub. No.: US 2026/0184741 A1
(43) Pub. Date: Jul. 2, 2026

(54) PEPTIDE BASED MEDICINES FOR TREATING CANCER

(71) Applicants: Dimas Jimenez Mendoza, Mexico City (MX); Paola Castillo Juarez, Mexico City (MX)

(72) Inventors: Dimas Jimenez Mendoza, Mexico City (MX); Paola Castillo Juarez, Mexico City (MX)

(21) Appl. No.: 19/563,693

(22) Filed: Mar. 11, 2026

Related U.S. Application Data
(63) Continuation-in-part of application No. PCT/IB2023/059165, filed on Sep. 15, 2023.

(51) Int. Cl. C07K 7/06; A61K 38/00; A61P 35/00; C07K 7/08

(52) U.S. Cl. CPC C07K 7/06; A61P 35/00; C07K 7/08; A61K 38/00

(57) ABSTRACT

The invention describes peptides with anticancer activity and exhibiting antagonistic activity against CD133 protein, which were obtained and designed by means of computer tools, evaluating physicochemical parameters of flexibility, hydrophilicity, hydrophobicity, antigenicity and total charge as described herein, in addition to the modeling of the protein, which allowed selecting 4 regions with the appropriate characteristics. After modeling the selected peptides, CD133 peptide-protein molecular docking was performed, where stable interactions were observed, even showing hydrogen bonds. Finally, the peptides of the invention showed anticancer effects against several cancer cell lines, including cancer cells resistant to conventional treatments such as triple negative breast cancer cells.

### CROSS-REFERENCE TO RELATED APPLICATIONS

This application is a continuation-in-part of International Application No. PCT/IB2023/059165, filed on Sep. 15, 2023, the contents of which are hereby incorporated by reference in their entirety.

### SEQUENCE LISTING

A Sequence Listing in the form of an XML file (entitled “DIM01-seql-000001.xml”, created on Sep. 15, 2023, and having a size of 5,685 bytes) is hereby incorporated by reference in its entirety.

### FIELD OF THE INVENTION

The invention relates to the technical field of genetic engineering and biomedicine, in particular to the selection and isolation of CD133 protein antagonist peptides that antagonize effects and limit the development of cancer. Pharmaceutical compositions including such peptides are provided for cancer therapy, in particular those resistant to conventional therapeutic treatments, such as chemotherapies.

### BACKGROUND OF THE INVENTION

Cancer, according to the World Health Organization (WHO), is defined as a broad group of diseases that can affect any part of the organism, having the characteristic of rapid multiplication of abnormal cells that extend beyond the usual limits, in addition to having the ability to invade adjacent parts or spread to different organs (metastasis) (WHO, 2018).

The National Cancer Institute of the United States of America (NCI, 2015) defines cancer as a set of related diseases in which cell growth and division occur in an uncontrolled manner. In this condition there is difference in specialization between cancer cells and normal cells, where, normal cells mature into very distinct cell types with specific functions, while cancer cells lack it. There are currently more than 100 types of cancer described.

Cancers almost always originate as a consequence of genetic alterations, the vast majority of which initiate in a single cell and are therefore monoclonal in origin. However, because a wide variety of genetic and epigenetic changes can occur in different cells within malignant tumors over time, most cancers are characterized by marked heterogeneity in the cell population. This heterogeneity significantly complicates the treatment of most cancers because there are likely to be subgroups of cells that will be resistant to treatment and thus survive and proliferate even if the majority of cells are eliminated (Clarck & Longo, 2012).

Cancer can be classified into benign neoplasm, which does not invade different tissues besides the local one, and malignant neoplasm or tumor, which has the characteristic capacity to invade tissues and the formation of metastasis or dissemination throughout the body.

In terms of incidence and mortality, the Pan American Health Organization (PAHO, 2020), positions cancer as the second leading cause of death in the Americas. In 2018, 9.6 million deaths were due to this disease, a figure that positions cancer as the second leading cause of mortality worldwide (WHO, 2020); about 70% of these cases were found in middle and low-income countries.

### Breast Cancer.

According to INEGI figures (October 2021), In Mexico 97,323 deaths have been attributed to malignant tumors, of which 7,880 were due to malignant breast tumors, equivalent to 8% of the above total. Likewise, it is shown that during 2019, 15,286 new cases of breast cancer were registered in the population aged 20 years and older, of which 167 corresponded to men and the rest (15,119) to women. The incidence rate in Mexico for this disease for 2019 was 18.55 new cases per 100,000 population aged 20 years and older (INEGI, 2021) (FIG. 1).

Breast cancer is described as the type of cancer that arises and develops from breast tissue cells, which can start in one or both breasts. Breast cancer can form from different parts of the organ, according to the site of origin it receives its name (ACS, 2022). In addition to the site of origin, this type of cancer can be classified according to the degree of severity it presents (Cleveland Clinic, 2022).

More specifically, breast cancer can be classified both biologically and clinically significant into subgroups according to histologic grade and type. Histological grade is defined as the degree of differentiation (e.g., tubule formation and nuclear polymorphism) and proliferative activity of a tumor which reflects its aggressiveness.

On the other hand, histologic type refers to the pattern of tumor growth. The histologic diversity of adenocarcinomas in the breast has long been a fascinating topic in the pathology community, which has identified specific morphologic and cytologic patterns that are directly associated with distinctive clinical presentations and/or diagnoses. These patterns are termed “histologic types”. The most frequent type of breast carcinoma are the so-called non-invasive ductal carcinoma-specific (IDC-NOS) or no special type (IDC-NST), which are exclusion criteria and comprise adenocarcinomas that do not exhibit sufficient features to warrant classification into one of the specific types (Weigelt, Geyer, & Reis-Filho, 2010).

Wellings and Jensen (1973) and Willings et al. (1975) questioned the belief of the available data that different histologic types of breast cancer arise from different microanatomic structures of the normal breast, demonstrating that the vast majority of invasive breast cancers and their in situ precursors are originated from the ductolobulbar terminal unit independently of histologic type.

Over the past 20 years, high-throughput microarray-based gene expression profiling has been widely applied to the study of breast cancer to unravel the molecular basis of biological features such as metastatic propensity or histologic grade, and to identify fingerprints associated with prognosis and response to therapy. In addition, analysis in the discovery of breast cancer types subjected to microarray-based expression profiling has led to a rediscovery at the molecular level of the long-appreciated morphological and clinical heterogeneity of breast cancer.

In an analysis by Perou et al. (2000), cDNA microarray analysis of 38 invasive breast cancers (36 invasive ductal carcinomas and 2 lobular carcinomas), 1 ductal carcinoma in situ, 1 fibroadenoma and 3 normal breast samples, and several biological replicates from the same patient, defined a list of “intrinsic” genes (genes that vary between tumors from different patients compared to samples from the same tumor/patient). This cluster analysis using the intrinsic gene list determined the division of the dendogram into ER-positive and ER-negative estrogen receptor-positive and ER-negative breast cancer groups, a robust feature when analyzing the transcriptome of breast cancers, and the existence of four molecular subtypes of breast cancer: luminal, normal, HER2 and basal type (Weigelt, Geyer, & Reis-Filho, 2010).

There are different types of treatment suggested for this pathology, among which are surgery, chemotherapy, radiotherapy, hormonal therapy, immunotherapy and, targeted drug therapy; the therapy suggested to the patient depends on several factors such as the location and size of the tumor, laboratory results and whether the cancer has spread to adjacent tissues or to other parts of the body.

Recently, the use of peptide therapy for the treatment of breast cancer has been reported. Peptides are short linear chains of amino acids (aa), usually less than 50 aa in length, which are often stabilized by disulfide bonds. They are designed by rational methods with high specificity to bind and modulate a protein interaction of interest. Various oncogenic protein interaction structures and patterns are available; and as such peptides can be designed as inhibitors of these interactions, for example, if a two-protein interaction is known, a peptide can inhibit this interaction, provided the sequence of the binding site is known. If the protein-protein binding site is unknown, a series of peptides overlapping the protein of interest are synthesized and their binding and inhibitory ability at the target site can be tested. Some of the important advantages and disadvantages of using therapeutic peptides are shown in Table 1 (Marqus, Pirogova, & Piva, 2017).

### Triple Negative Breast Cancer.

Triple negative breast cancers (TNBC) are defined as tumors that have an absence of estrogen receptor (ER), progesterone receptor (PR or PgR), and HER2 (human growth factor receptor 2) expression (FIG. 3) (Foulkes, Smith, & Reis-Filho, 2010), and are most commonly found in people who have a mutation in the BRCA1 gene (tumor suppressor gene) (ACS, 2022).

TABLE 1
Advantages and disadvantages of therapeutic peptides.

| Advantages | Disadvantages |
|---|---|
| High action potential | Metabolic instability |
| High specificity and target selectivity | Poor membrane permeability |
| Wide range of whites | Low oral bioavailability |
| Low toxicity | Rapid excretion |
| Few side effects | Low solubility |
| Low bioaccumulation | High manufacturing cost |
| High biological and chemical diversity | Little activity |

Basal-like cancers constitute one of the four intrinsic subgroups of breast cancer, the existence of which was revealed by microarray-based expression profiling studies. This subgroup is characterized by the absence or low levels of ER expression, an absence or overexpression of HER2 and expression of genes usually detected in basal or myoepithelial cells in normal breast tissue cells. Some types of breast cancers meet the characteristics of both triple negative and basal type breast cancers. Currently there is no international acceptance for the definition of basal-like cancers, because most basal cancers are also TNBC and most TNBC cancers (approximately 80%) are also basal-like (Foulkes, Smith, & Reis-Filho, 2010).

Individuals with TNBC do not benefit from endocrine or trastuzamab treatments. Chemotherapy is currently the treatment of choice for the systemic condition, where patients who undergo this type of therapy have greater post-chemotherapy side effects, improving the diagnostic outcome compared to patients with the ER-positive subtype.

The use of neoadjuvant chemotherapy prior to surgery has been shown in a minority of patients with TNBC to be more effective in treatment, in contrast, the majority of patients show residual disease after treatment, this suggests that there is a subgroup of patients with TNBC whose tumors are extremely sensitive to chemotherapy.

There is currently no standard treatment for TNBC, and this must be selected as for other subtypes of breast cancers (Foulkes, Smith, & Reis-Filho, 2010).

Accordingly, it is necessary to continue with the development of therapeutic solutions that allow the effective treatment of cancer, thus increasing the possibilities of treatment for cancer that is resistant to the treatments known to date.

### BRIEF SUMMARY OF THE INVENTION

The invention describes peptides with antineoplastic activity exhibiting antagonistic activity against the CD133 protein, which were obtained and designed by means of computer tools, evaluating physicochemical parameters of flexibility, hydrophilicity, hydrophobicity, antigenicity and total charge as described herein, in addition to the modeling of the protein, which allowed selecting 4 regions with the appropriate characteristics. After modeling the selected peptides, CD133 peptide-protein molecular docking was performed, where stable interactions were observed, even showing hydrogen bonds. The peptides of the invention showed anticancer effects against several cancer cell lines, including cancer cells resistant to conventional treatments such as triple negative breast cancer cells.

It is one of the objectives of the invention to provide peptides that antagonize CD133 glycoprotein activity and thus counteract cancer stem cell proliferation and tumor differentiation.

In another aspect of the invention, the design of peptides based on the study of the molecular structure of the CD133 glycoprotein is provided.

In another aspect of the invention evidence is provided for the ability of the peptides described herein to limit cancer cell proliferation.

Another aspect relates to pharmaceutical compositions containing at least one of the peptides, designed for a preferably parenteral route of administration.

It is an embodiment of the invention to provide a medicament capable of preventing the development or differentiation to the tumor phenotype.

It is an embodiment of the invention to provide a medicament for aiding in the treatment of cancerous tumors.

It is an embodiment of the invention to provide a drug for aiding in the elimination of drug resistant CSCs (Cancer Stem Cells).

It is another object of the invention to provide peptides, specifically the peptides ABCP-1, ABCP-2, ABCP-3 and ABCP-4, capable of interacting with the transmembrane protein CD133 when over expressed in cancer cells, and limiting its role in tumorogenesis and tumor progression.

It is object of the invention to provide the peptide ABCP-1 (SEQ. ID. NO. 1) and pharmaceutical compositions containing them for reducing cell viability of cells from breast, bone, brain and larynx cancer.

It is object of the invention to provide the peptide ABCP-4 (SEQ. ID. NO. 4) and pharmaceutical compositions containing them for reducing cell viability of cells from breast, bone, brain and larynx cancer.

Embodiments of the invention are the possibility of formulating medicaments comprising at least one of the peptides ABCP-1 (with the sequence SEQ. ID. NO. 1) or of the peptide ABCP-4 (with the sequence SEQ. ID. NO. 4), with the precise doses to act as anticancer and/or to prevent, diminish or fight cancerous tumors, the effective doses for patients can be obtained through studies designed by persons skilled in the art and the patients can be mammals including humans.

Furthermore, it is modality of the invention any combination of the peptides jointly or separately, with other active principles used in cancer therapy.

### Abbreviations.

Aa: amino acids,
BCBL: Breast Cancer Basal Like (Breast Cancer Basal Like),
ER: Estrogen receptor,
HER2: Human growth factor receptor 2,
PR or PgR: Progesterone receptor,
CSC: Cancer Stem Cells.

### BRIEF DESCRIPTION OF THE FIGURES

FIG. 1. Breast cancer incidence in the population aged 20 years and older.

FIG. 2. Non-triple negative (left) and triple negative (right) breast cancer cells are shown.

FIG. 3. The topographic model of Prominin-1/CD133 is shown.

FIG. 4. Various functional roles of CD133 as a regulator of CSC are shown.

FIG. 5. tertiary structure of the CD133 protein, using the I-Tasser server.

FIG. 6. The ABCP-1 peptide of the invention is shown, which corresponds to amino acids 40-50 of prominin-1 isoform 1 precursor (e.g., found in NCBI Reference Sequence: NP_006008.1).

FIG. 7. The evaluated physicochemical parameters of the CD133 protein consensus sequence are shown. A) hydrophilicity prediction, determined with IEDB; B) flexibility determination with IEDB; C) total charge determination with EMBOSS charge; D) antigenicity prediction, determined with IEDB; E) hydrophobicity prediction determined with EXpasy's ProtScale, using the Kyte & Doolittle scale.

FIG. 8. The ABCP-2 peptide of the invention is shown, which corresponds to amino acids 559-570 of prominin-1 isoform 1 precursor (e.g., found in NCBI Reference Sequence: NP_006008.1).

FIG. 9. The ABCP-3 peptide of the invention is shown, which corresponds to amino acids 589-599 of prominin-1 isoform 1 precursor (e.g., found in NCBI Reference Sequence: NP_006008.1).

FIG. 10. The ABCP-4 peptide of the invention is shown, which corresponds to amino acids 634-643 of prominin-1 isoform 1 precursor (e.g., found in NCBI Reference Sequence: NP_006008.1).

FIG. 11. The electrostatic and hydrogen-bonding interactions between the ABCP-1 peptide and the CD133 protein are shown.

FIG. 12. The electrostatic and hydrogen bonding interactions between the ABCP-2 peptide and the CD133 protein are shown.

FIG. 13. The electrostatic and hydrogen bonding interactions between the ABCP-3 peptide and the CD133 protein are shown.

FIG. 14. The electrostatic and hydrogen bonding interactions between the ABCP-4 peptide and the CD133 protein are shown.

FIG. 15. The percentage of survival in different cell lines exposed to the ABCP-1 and ABCP-4 peptides of the invention at an initial concentration of 40.8 mg for 48 h is shown. Cell lines: U87-MG, HEp2 and U2-OS, controls WITHOUT peptide, and against cells of non-neoplastic origin (Vero). The graph shows the mean±SEM. Analyzed by two-factor ANNOVA with a post Holm-Sidak test. (*) p<0.05 with respect to control.

FIG. 16. The effect of ABCP-1 (A,C) and ABCP-4 (B,D) peptides on MCF-7 line cell viability and percent survival is shown. The graphs show the mean±SEM. A) and B) were analyzed by a 3-parameter nonlinear regression, determining the CI₅₀ at each time, while graphs C) and D) were analyzed by a two-factor ANOVA with a post hoc Dunnet's test (*) p<0.05 with respect to the control. The reading at 24 hr (black), 48 hr (red) and 72 hr (blue), as well as cell control (green) are shown.

FIG. 17. The effect of ABCP-1 (A,C) and ABCP-4 (B,D) peptides on MDA-MB231 line cell viability and percentage survival is shown. A) and B) were analyzed by a 3-parameter nonlinear regression, determining the CI₅₀ at each time, while graphs C) and D) were analyzed by a two-factor ANOVA with a post hoc Dunnet's test (*) p<0.05 with respect to the control.

FIG. 18. Diameter of the tumor developed in the breast cancer murine model. Once the administration protocol for ABCP-1, ABCP-4, or the combination of both peptides was completed, the tumor mass developed by each specimen was measured (ex vivo measurement), with a follow-up period of 14 days.

FIG. 19. Reduction in the diameter of the tumor developed in the breast cancer murine model. Once the administration protocol for ABCP-1, ABCP-4, or the combination of both peptides was completed, the tumor mass developed by each specimen was measured (ex vivo measurement), with a follow-up period of 14 days. The mice treated with the peptides ABCP-1, ABCP-4, or the mixture of both showed a statistically significant reduction in the diameter of the developed tumor (p<0.05, one-way ANOVA).

FIG. 20. Reduction in tumor volume in the breast cancer murine model. After completing the administration protocol of the peptides ABCP-1, ABCP-4, or the combination of both, the tumor mass developed by each specimen was measured (ex vivo measurement) after a follow-up period of 14 days. The mice treated with the peptides ABCP-1, ABCP-4, or the mixture of both showed a reduction in tumor volume.

FIG. 21. Percentage of reduction in tumor diameter in a murine model. After completing the administration protocol of the peptides ABCP-1, ABCP-4, or the combination of both, the tumor mass developed in each specimen was measured (ex vivo measurement) after a follow-up period of 14 days. Mice treated with the peptides ABCP-1, ABCP-4, or the mixture of both showed a reduction in tumor volume.

FIG. 22. Percentage of reduction in tumor volume in a breast cancer murine model after completion of the ABCP-1, ABCP-4, or combination administration protocol. The tumor mass developed by each specimen was measured (ex vivo measurement), with a follow-up period of 14 days. Mice treated with the peptides ABCP-1, ABCP-4, or a mixture of both showed a reduction in tumor volume.

FIG. 23. Weight of mice treated with ABCP-1 and/or ABCP-4 peptides. Once tumor development was induced, the mice were weighed daily until the end of the trial (day 14).

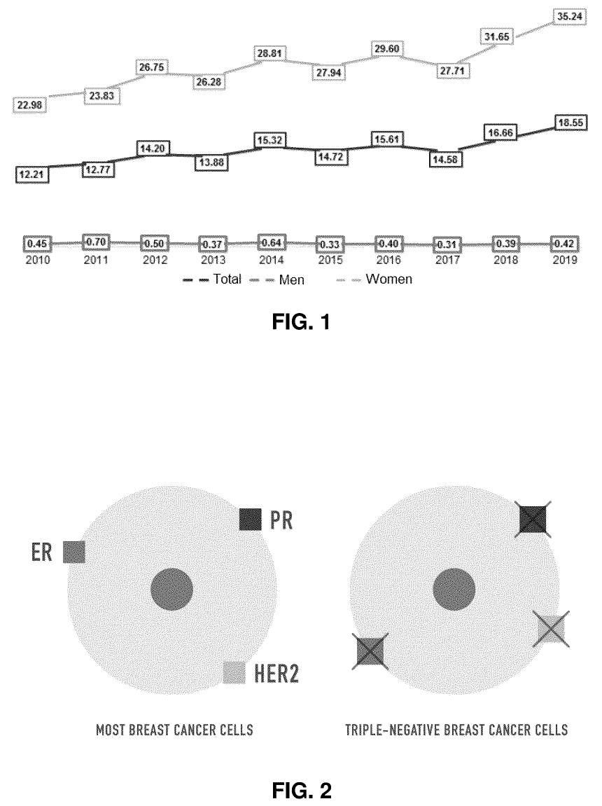

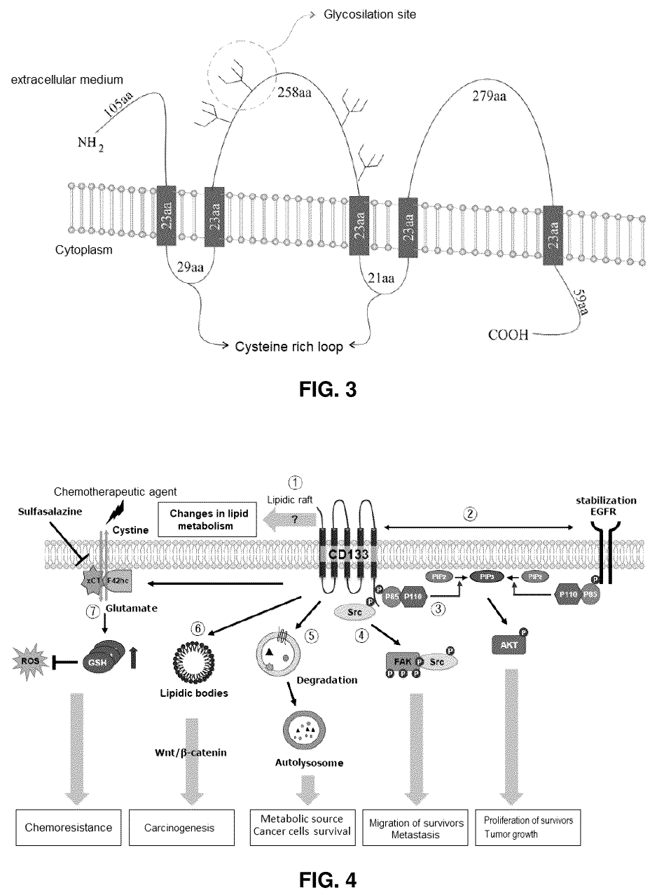

### DETAILED DESCRIPTION OF THE INVENTION

The invention describes peptides with antineoplastic activity exhibiting antagonistic activity against the CD133 protein, which antagonize its effects and significantly limit the development of cancer. The peptides of the invention were obtained and designed using computer tools, evaluating physicochemical parameters of flexibility, hydrophilicity, hydrophobicity, antigenicity and total charge as described herein, in addition to the modeling of the protein, which allowed the selection of 4 regions with the appropriate characteristics. After modeling the selected peptides, molecular docking of peptides of the invention-protein CD133 was performed, where stable interactions were observed, even showing hydrogen bonds. The peptides of the invention were able to significantly inhibit the growth of various cancer cell lines, including triple negative breast cancer cells that commonly exhibit resistance to therapeutic treatments known prior to the present invention.

CD133 protein. Prominin-1, also known as CD133, contains 5 transmembrane domains with two large glycosylated extracellular loops and two smaller intracellular loops comprising 250 and 20 amino acids respectively. It has a molecular weight of 115/120 kDa and comprises 850-865 amino acids, where the N-terminus is exposed to the extracellular milieu and the C-terminus is exposed to the cytoplasm (intracellular milieu). The human gene encoding prominin-1 is located on chromosome 4 and contains at least 37 exons. It is of pentaspan glycoprotein nature that binds to membrane cholesterol (lipid microdomain), specifically localizes to plasma membrane protrusions and accumulates in membrane lipid microdomains (FIG. 24) (Barzegar-Behrooz, Syahir, & Ahmad, 2019).

The gene encoding CD133, prominin 1 (PROM1), is located on chromosome 4 in humans and on chromosome 5 in mice, where there is approximately 60% homology between primates and rodents. Transcription in humans of this gene is mediated by five alternative promoters, three of which are located on CpG islands and are partially regulated by methylation. These promoter regions often result in alternative splicing of CD133 mRNA, resulting in variable structures with potentially unique functions.

Several molecular studies have suggested a potential role for CD133 in cell maintenance and cell fate, having stem cell-like properties.

Hypoxia in stem cells and the tumor microenvironment have demonstrated CD133 expression through upregulation of factor 1α (HIF-1α). CD133 has been postulated to identify CSC populations in number of solid tumor types, including various forms of brain, prostate, colon, lung, hepatocellular carcinoma and ovarian cancer. In these studies, CD133 expression on CSCs exhibited self-renewal potential and the ability to regenerate a histologically similar tumor mass after transplantation in immunodeficient mice (Glumac & LeBeau, 2018).

Cancer stem cells (CSCs) are a small population of cells in tumor tissue that are capable of inducing tumor initiation and differentiation. In myeloid leukemia cells, these CSCs were shown to have the ability to generate tumors in NOD/SCID mice after inoculation. In 2003, the existence of CSCs was demonstrated in a solid breast cancer tumor. These CSCs have the ability to initiate metastasis and possess resistance to chemotherapy and radiotherapy. Due to this capacity, they were considered to be the cause of recurrence in this type of cancer.

The CD133 glycoprotein was identified from mouse neuroepithelial stem cells and human hematopoietic stem cells. CD133 expression was not limited to normal stem cells, but has also been discovered as a marker in tumor cells of different types. These CD133+ cells have shown three main characteristics: 1) self-renewal capacity, 2) tumor proliferation and differentiation, and 3) tumor formation. Previous studies showed that the presence of CD133 is closely related to drug resistance genes, e.g., ABCG1 and ABCG2 (Jae-Woo, Yeonhwa, Se-Hyuk, Joon, & Heng, 2017). The pathways seen to be related to CD133 in different cancer types are shown in Table 2 and FIG. 4.

TABLE 2
Mechanisms related to CD133 and cancer

| Related mechanism | Type of cancer |
|---|---|
| via pi3k-akt | Hepatocellular carcinoma (hcc) |
| | Neuroblastoma |
| | Glioma, colon cancer |
| VIA SRC-FAK | Colon cancer |
| EGFR | Pancreatic cancer |
| Autophagy | hcc |
| | Glioma |
| Lipid metabolism | Colon cancer |
| | Neuroblastoma |
| Oxygen species | Prostatic carcinoma |
| reactive (ros) | Colon cancer |
| | Gliomas |
| | hcc |

CD133 controls the acquisition of properties in CSCs by regulating signaling molecules and changing tumor metabolism, where: 1) the cholesterol binding protein, CD133, can change lipid metabolism by induction of a lipid raft, through recomposition of cholesterol in the cell membrane, 2) CD133 increases EGFR-AKT signaling through EGFR stabilization, where EGFR stabilization may be due to direct binding to CD133, 3) PI3K-AKT signaling is activated by the interaction between CD133 and the P85 subunit, 4) Src kinase is phosphorylated by physical interaction with CD133, where in turn, FAK is activated by Scr which induces tumor migration and survival, 5) CD133 induces autophagosome formation through release from the membrane into the cytoplasm, 6) CD133-induced lipid bodies positively regulate tumor-promoting signaling via Wnt/β-catenin, and 7) CD133+ cells possess resistance to oxidative stress through positive regulation at the GSH level (Jae-Woo, Yeonhwa, Se-Hyuk, Joon, & Heng, 2017).

It has been observed that CD133+ prostate cancer cells, when induced with docetaxel (antineoplastic drug), showed significant post-treatment resistance, which was inoculated at concentrations of 2 and 4 nM, and when compared with CD133-cells of the same type of cancer. Furthermore, in another trial, when exposed to radiotherapy using a gamma irradiator of cells applying doses of 4 and 8 Gy (Gray; J/kg), higher post-irradiation resistance was observed in CD133+ cells compared to CD133− (Kanwal, Shukla, Walker, & Gupta, 2018).

In metastatic ovarian cancer, CD133 mRNA upregulation is regulated by the transcription factor ARID3B. In CD133-deficient cells with overexpressed ARID3B, faster death was observed in xenograft, tumor-caused mice using CD133+ ARID3B+ cells as a comparison (Liou, 2019). CD133 expression has been associated with increased lymph node metastasis, upregulation of vascular endothelial growth factor C expression and 5-year decreased survival in patients with pancreatic cancer (Roy, et al., 2018).

Some CD133-derived peptides with tumor inhibitory capabilities have now been described such as the one described in patent application CN111529690A where a peptide fragment 1-108 of the human CD133 protein is disclosed, which is applied to temozolomide chemotherapy to treat glioma as an intensive chemotherapy drug.

Another approach in the use of CD133-derived peptides is as antigens for the purpose of generating an immune response against CD133-expressing tumors, this is disclosed in application CN106581668A, which describes a composition of four antigenic peptides, namely STEAP1 antigenic peptide, PSM antigenic peptide, DKK1 antigenic peptide and CD133 antigenic peptide. Experimental results show that when the antigenic peptide composition is adopted, dendritic cell maturation can be effectively induced, antigen presentation ability is improved, specific cytotoxicity killer cells can be generated, tumor killing efficiency is improved, and tumor growth can be effectively controlled.

Notwithstanding the foregoing, the state of the art does not describe or disclose CD133 protein antagonist peptides of reduced size with the characteristics of those disclosed herein, capable of antagonizing the effects of said protein and exhibiting antineoplastic effects.

In one embodiment, the invention comprises pharmaceutical compositions with anticancer activity comprising the peptides of the invention as active principles.

The results of the present invention show that the peptides described herein are useful as a new therapeutic alternative for the treatment of cancer patients, including those cancers that are resistant to conventional therapeutic treatments, such as, for example, triple negative breast cancer, whereby it is part of the modalities of the invention to use the peptides ABCP-1 (SEQ. ID. No. 1), ABCP-2 (SEQ. ID. No. 2), ABCP-3 (SEQ. ID. No. 3) and/or ABCP-4 (SEQ. ID. No. 4) as such or mixtures thereof for obtaining compositions for pharmaceutical use comprising the same for the treatment of said conditions.

Therefore, it is one of the objectives of the invention to provide pharmaceutical compositions comprising the peptides ABCP-1 (SEQ. ID. No. 1), ABCP-2 (SEQ. ID. No. 2), ABCP-3 (SEQ. ID. No. 3) and/or ABCP-4 (SEQ. ID. No. 4), either together or separately, together with pharmaceutically acceptable excipients suitable for administration to the patient requiring them. It is modality of the invention to adapt the active principles for use in pharmaceutical compositions for enteral administration, parenteral administration and topical use, including inhalation. The effective doses for the patient of the active principle will also be adjusted in accordance with the preclinical and clinical studies, but taking as a basis the findings of the present invention.

Examples of pharmaceutically acceptable excipients accompanying the active principle of the invention are for example, for oral administration as tablets or tablets, agents comprising for example diluents, binders, stabilizers, bulking agents, thickening agents, such as povidone, microcrystalline cellulose, lactose, etc., disintegrating agents such as for example cross-linked carboxymethyl cellulose, surfactants such as for example sodium lauryl sulfate, lubricating or sliding agents such as for example magnesium stearate, colloidal silicon dioxide, etc., wherein said excipients may be formulated for preferably slow or prolonged release for a systemic effect.

Solutions for intravenous or intraperitoneal administration of the active ingredient can be prepared by first dissolving them in an organic solvent such as DMSO, ethanol, or dimethylformamide and subsequently in aqueous buffers, such as PBS.

Particular preference is given to pharmaceutical forms designed for localized administration to the site where the cancer to be treated is present, where liquid or solid compositions can be formulated, suitable for local administration for example, and whose excipients can be selected for example from components compatible with said pharmaceutical form, for example of a lipidic or peptidic nature, or peptidomimetics, known in the state of the art as non-immunogenic, and which preferably can be bound to the active principles of the invention to improve their bioavailability; propellant agents such as, for example, propane, butane, or permissible chlorofluorocarbons; pH regulators such as, for example, sulfuric acid; chelating agents such as, for example, EDTA. The active ingredients can also be formed into micronized particles contained in gelatin capsules or other systems known in the technical field, which help to release the active ingredient to its target site of action, for example, by means of solid dosage forms such as tablets or dragees, including those formulated for prolonged release.

According to the present invention, the compositions described herein may be obtained by combining the peptides ABCP-1 (SEQ. ID. No. 1), ABCP-2 (SEQ. ID. No. 2), ABCP-3 (SEQ. ID. No. 3) and/or ABCP-4 (SEQ. ID. No. 4) with pharmaceutically compatible vehicles known in the art, in the amounts and/or concentrations that correspond as described herein, and compounds known in the art may be included to obtain such compositions. Likewise, the administration of such compositions, may be made depending on the conditions of the patient, which will determine the doses and frequency of administration necessary to achieve an effective treatment of the ailment in each particular case.

In another embodiment of the invention, methods of treatment for cancer patients, including those cancers which are resistant to conventional therapeutic treatments, such as, for example, triple negative breast cancer, are included, comprising administering to the cancer patient the pharmaceutical compositions comprising the peptides of the invention, in the amounts, dosage frequency and time the treatment is indicated by the treating physician. As can be seen from the results described herein, said compositions will have an anti-cancer effect on the affected cancer patients, including those suffering from cancer resistant to conventional therapeutic treatments.

In yet another embodiment of the invention, nucleic acid sequences coding for the peptides described herein are included which are useful for the production of said peptides by methods of molecular biology and genetic manipulation known in the art, which include cloning said sequences into known molecular vehicles so that said vehicles express the peptides of interest in suitable culture conditions under the control of suitable expression promoters, wherein said peptides are finally obtained in significant quantities to be subsequently purified by methods known in the art for subsequent use in obtaining the pharmaceutical compositions described herein and their potential administration to patients suffering from cancer. One skilled in the art will recognize that such methods are feasible from the coding nucleic acid sequence of for the peptides of the invention described herein.

### Development of the Peptides of the Invention.

The amino acid sequences of the seven CD133 protein isoforms were downloaded from the NCBI GenBank database (https://www.ncbi.nlm.nih.gov/protein), and from UniProt (http://www.uniprot.org/) with identification key NP_006008.1 and 043490, respectively, both in FASTA format. The peptides of SEQ. ID. NOs 1-4 were derived from a consensus sequence between the sequence of NP_006008.1 (https://www.ncbi.nlm.nih.gov/protein) and 043490 (http://www.uniprot.org/).

Subsequently, the downloaded amino acid sequences were aligned multiple times to search for conserved regions using the Aling sequences with COBALT tool (https://www.ncbi.nlm.nih.gov/tools/cobalt/re_cobalt.cgi) which performs the alignment using the conserved domain and local sequence similarity information.

From the result of the multiple alignment, a consensus amino acid sequence for the CD133 protein was obtained using GeneDoc (http://www.nrbsc.org/gfx/genedoc/), which integrates structural information, searches for conserved regions and compares sequences in terms of percentage identity. With this sequence, the tertiary structure modeling of the protein and the physicochemical analysis were performed.

### Tertiary Structure Prediction of CD133 Protein.

a. The prediction of the three-dimensional structure of the CD133 glycoprotein was performed using the I-Tasser server,
b. Protein refinement was performed using the Mod-Refiner server,
c. Protein visualization was performed using the Chimera program,
d. Some physicochemical parameters of the consensus amino acid sequence of the CD133 protein were evaluated using a window of 6, 7 and 9 amino acids. Different servers were used as explained below.

For hydrophobicity, ProtScale with Kyte-Doolitle scale available in ExPASy was used, which allows the representation in a two-dimensional plot of a profile induced by any amino acid scale within a selected protein. (http://web.expasy.org/protscale/) with Kyte-Doolitle scale (available in ExPASy). To generate data for a plot, the protein sequence is scanned with a sliding window of a given size. At each position, the average scale value of the amino acids within the window is calculated, and that value is plotted for the midpoint of the window.

The Kyte-Doolitle scale is based on a series of experimental observations derived from the literature. The program uses a moving segment approach that continuously determines the average hydrophobicity (hydrophobicity or hydrophilicity state) within a segment of predetermined size as it moves through the sequence. Consecutive values are plotted from the amino to the carboxyl-terminal end (Kyte, J. & Doolittle, R., 1982).

For hydrophilicity, linear epitope prediction available from IEDB analysis resources (http://tools.immuneepitope.org/bcell/) was used, with Parker scaling, which allows the representation in a two-dimensional plot of a profile induced by any amino acid scale within a selected protein. To generate a plot, the protein is scanned into a predetermined amino acid sliding window, where the average scale value of the amino acids within the window is calculated and that value is plotted for the midpoint of the window.

The Parker scale is based on the results obtained from the retention times of 20 synthetic amino acid models during high-performance liquid chromatography (HPLC), where these results are used in a mathematical algorithm that assigns a value to each amino acid as the protein sequence progresses. The values are plotted from the amino to the carboxyl-terminal end (Parker J M et al. 1986).

For the antigenicity parameter, IEDB analysis resources were used, with the Kolastar & Tongaonkar scale, which allows the representation in a two-dimensional plot of a profile induced by any amino acid scale within a selected protein. To generate a plot, the protein is scanned into a predetermined amino acid sliding window, where the average scale value of the amino acids within the window is calculated and that value is plotted for the midpoint of the window (http://tools.immuneepitope.org/bcell/), with the Kolastar & Tongaonkar scale. The Kolastar & Tongaonkar scale is based on a semi-empirical method that makes use of the physicochemical properties of amino acid residues and their frequencies of occurrence in experimentally known epitope segments, where these data are contained in a mathematical algorithm that assigns a value to each amino acid as the protein sequence progresses. The values are plotted from the amino to carboxyl-terminal end (Kolastar A. & Tongaonkar P. 1990).

The flexibility parameter, available in the IEDB analysis resources, was also evaluated with the Karplus & Schulz scale, which allows the representation in a two-dimensional plot of a profile induced by any amino acid scale within a selected protein. To generate a plot, the protein is scanned into a predetermined amino acid sliding window, where the mean scale value of the amino acids within the window is calculated and that value is plotted for the window the midpoint of (http://tools.immuneepitope.org/bcell/) with Karplus & Schulz scale.

The flexibility scale is based on the mobility of protein segments based on knowledge of the temperature B-factors of the α-carbons of 31 proteins of known structure. The calculation is performed supported by a flexibility scale where the center is the first amino acid of a 6 amino acid long window employing three scales to describe flexibility instead of only one. The values are plotted from the amino to the carboxyl-terminal end (Karplus P. & Schulz G. 1985).

For the total charge parameter we use EMBOSS Charge (available on the server), which allows the representation in a two-dimensional average charge diagram of the amino acid sequence of the protein, while it is scanned in a sliding window of specific length, assigning a value to each amino acid and making the graph (http://www.bioinformatics.nl/cgi-bin/emboss/charge). The Charge algorithm uses the “charge” column of a data file (Eamino.dat) of amino acid properties, then calculates the average charge which is reflected in a plot (Bleasby A. 2001).

In accordance with the present invention, regions of the protein shown to be hydrophilic, flexible, chargeable and antigenic were those selected for modeling peptides. Such regions comprise amino acids 40-50, 559-570, 589-599 and 634-643.

We proceeded to the tertiary structure modeling of the four selected peptides using the PEP-FOLD server, to subsequently perform the refinement of the tertiary structure of the peptides using the Mod-Refiner server. Peptide visualization was performed using Chimera 1.12.

Molecular docking was performed using AutoDock vina, version 1.1.2, with the four peptides modeled in PDB format, and the CD133 protein modeled in its tertiary structure, in PDB format. Molecular docking was performed by challenging each peptide independently with the CD133 protein.

### Results of Tertiary Structure Prediction of CD133 Protein.

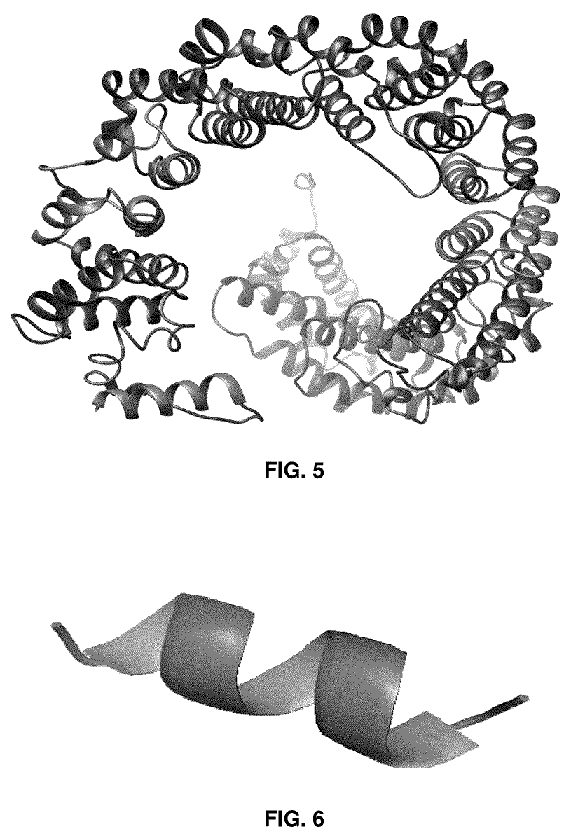

The result of the tertiary structure prediction of the CD133 protein is presented in FIG. 5. The protein is shown in a spiral shape, starting from amino acid 1 to 827, i.e. from amino to carboxyl terminal. The regions marked in blue, comprise amino acids 1-19 corresponding to the signal peptide; 109-178 first transmembrane and cytoplasmic region; 434-507 second transmembrane and cytoplasmic region; and 793-827 third and last transmembrane and cytoplasmic region.

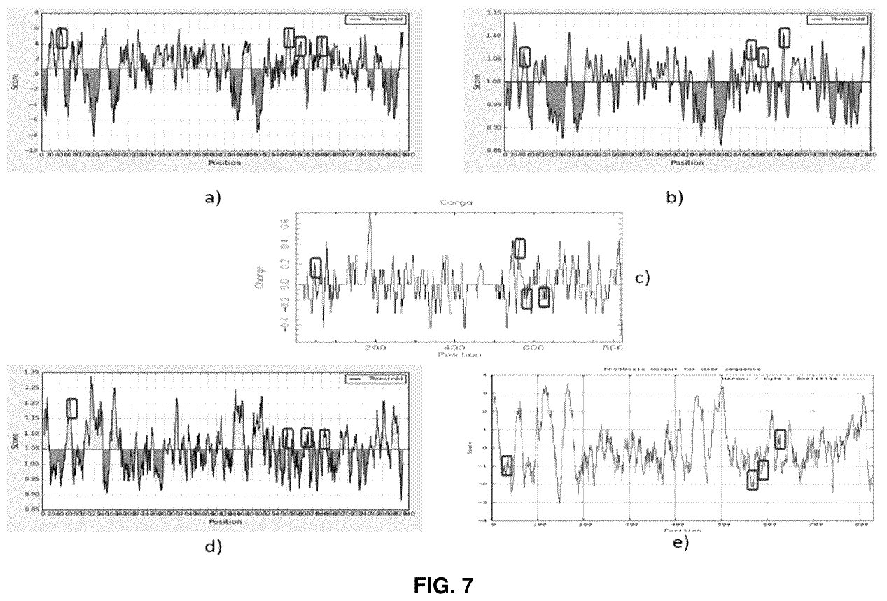

The results obtained from the physicochemical analysis of the CD133 protein consensus sequence were based on its primary structure, which comprises 827 amino acids, using bioinformatics programs. Each determination performed is independent, since as described above, a different server was used for one, therefore, the scales and/or scores are not the same, however, the graphs shown here share only the characteristic of presenting at the top of the graph, above the cut-off line, the attribute evaluated as the case may be, with the exception of the total charge determination. The results are shown in FIG. 7, where it can be observed:
A) Predicted hydrophilicity, determined with IEDB using the Parker scale (hydrophilic regions are indicated on the horizontal line, values >0.709 indicate hydrophilicity);
B) Determination of flexibility with IEDB using the Karplus & Schulz scale (values of >1.0 indicate flexibility, above the horizontal line);
C) Total load determination with EMBOSS charge;
D) Antigenicity prediction, determined with IEDB, using the Kolastar & Tongaonkar scale (values >1.047 indicate antigenicity, above the horizontal line);
E) Hydrophobicity prediction determined with EXpasy's ProtScale, using the Kyte & Doolittle scale. The amino acid regions 40-50, 559-570, 589-599 and 634-643 of the protein indicated in the graphs enclosed by a red box are those shown to be hydrophilic, flexible, charged and antigenic, which were selected to model the peptides of the invention.

It should be mentioned that the regions comprising amino acids 109-178, 434-507 and 793-827 were excluded since transmembrane and cytoplasmic sites are located in those regions, although the analysis was performed on the entire protein. For the purposes of the present invention, these areas proved not to be useful.

The hydrophilicity analysis was carried out using the IEDB program, where hydrophilic regions can be seen above the cut-off line, although several regions that present this characteristic can be observed (FIG. 7), only those whose value was the maximum and was present in at least 6 continuous amino acids were chosen; these regions correspond to amino acids 22-28, 40-50, 220-226, 264-269, 559-570 and 589-594.

Flexibility was evaluated using the tool available in the IEDB analysis resources, with the Karplus & Schulz scale. In this analysis, FIG. 7 shows the regions that are highly flexible, in yellow color, above the cut line; although several regions that present this characteristic can be observed, only those whose resulting value was the maximum and was present in at least 6 continuous amino acids were chosen, which include amino acids 21-27, 40-51, 83-95, 282-304, 350-377, 563-570, 589-599, 637-643, 663-677.

Antigenicity was evaluated using the tool available in the IEDB analysis resources, with the Kolastar & Tongaonkar scale. In this analysis, FIG. 7 shows the regions that are highly antigenic in yellow color, above the cut-off line; although several regions that present this characteristic can be observed, only those whose resulting value was the maximum and was present in at least 6 continuous amino acids were chosen, which comprise amino acids 21-27, 40-51, 83-95, 282-304, 350-377, 563-570, 589-599, 637-643, 663-677.

The charge was evaluated using the EMBOSS charge program. In this analysis, FIG. 7 shows the regions that present charge, both positive and negative, starting from zero as a cut-off point. The minimum charge was-3.067 at amino acid 148, while the maximum charge was +3.578, however, these regions are found in the cytoplasm comprising the amino acids; although, on the other hand, it can be observed that the protein has regions with both positive and negative charge.

The results of the hydrophobicity analysis were obtained using the EXpasy program, in the ProtScale server, with the Kyte & Doolittle scale, where in FIG. 7 hydrophobic regions above the value of zero can be seen, also observing that these regions are exactly opposite to those presented by the hydrophilicity.

### Modeled Peptides of the Invention.

As a result of the described analyses, the following peptides were modeled:

ABCP-1. This peptide corresponds to the region comprising amino acids 40-50 of prominin-1 isoform 1 precursor (e.g., found in NCBI Reference Sequence: NP_006008.1) (FIG. 6); it can be observed that most of its structure is composed of an alpha-helix, however, at the ends it presents a kind of loop that gives it certain anchoring and flexibility characteristics. Its amino acid sequence is shown below:
NYETQDSHKAG (SEQ. ID. No. 1)

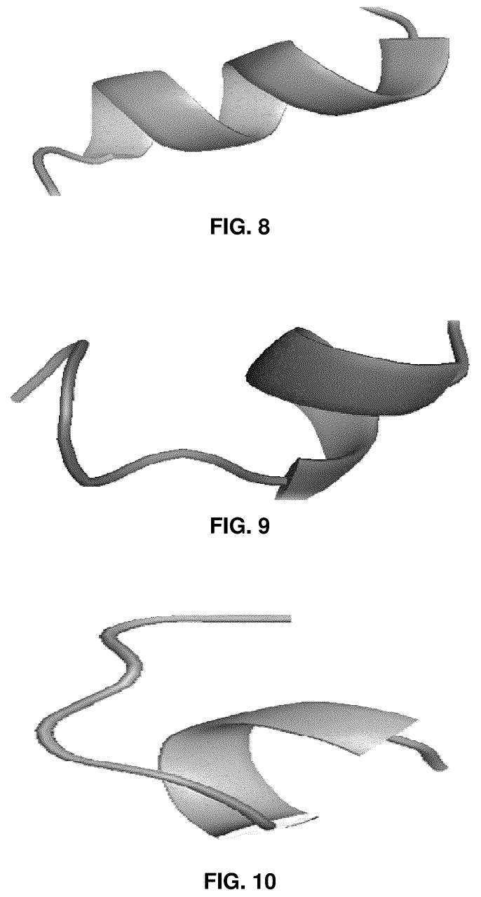

ABCP-2. This peptide corresponds to the region comprising amino acids 559-570 of prominin-1 isoform 1 precursor (e.g., found in NCBI Reference Sequence: NP_006008.1) (FIG. 8); it can be observed that most of its structure is composed of an alpha-helix (like ABCP-1); however, at the ends the structure it presents has been found to be even more flexible, and therefore, it would have a more stable anchorage. Its amino acid sequence is shown below:
VYSDCKKNRGTY (SEQ. ID. No. 2).

ABCP-3. This peptide corresponds to the region comprising amino acids 589-599 of prominin-1 isoform 1 precursor (e.g., found in NCBI Reference Sequence: NP_006008.1) (FIG. 9); in this peptide the arrangement of its structure is different from the previous peptides, since it is observed that half of the peptide corresponds to an alpha-helix, while the other half is a beta-folded one. Its amino acid sequence is shown below:
EHTGSISSELE (SEQ. ID. No. 3).

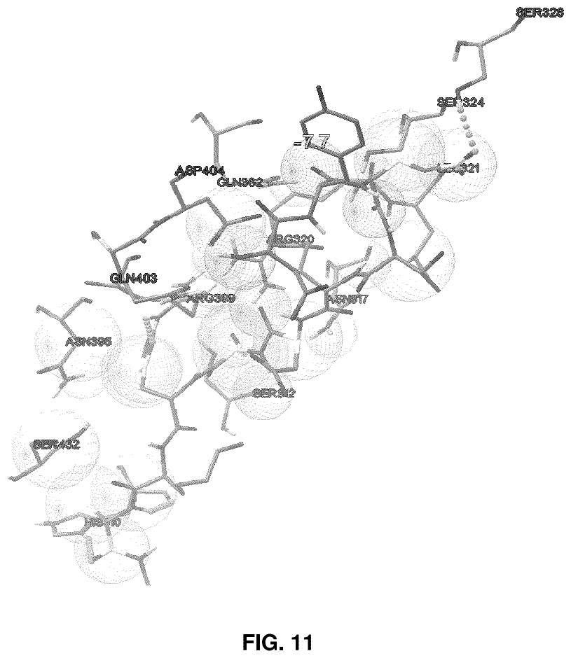

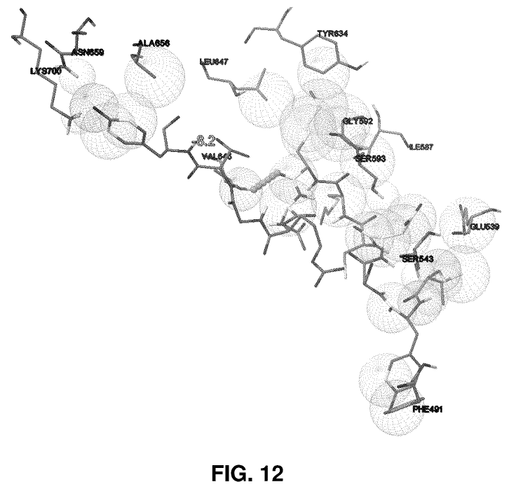

ABCP-4. This peptide corresponds to the region comprising amino acids 634-643 of prominin-1 isoform 1 precursor (e.g., found in NCBI Reference Sequence: NP_006008.1) (FIG. 10); in this peptide the arrangement of its structure is different from the previous peptides, since it is observed that less than half of the peptide corresponds to an alpha-helix, while the remaining part is a beta-folded, it also has a small flexible structure similar to that which appears in the ABCP-1 and ABCP-2 peptides, which confers a better interaction and anchorage. Its amino acid sequence is shown below:
YLAQTGKSPA (SEQ. ID. No. 4).

### Molecular Docking.

Molecular docking was carried out with the complete amino acid sequence of CD133 against each of the modeled peptides, however, in the results shown below, data from those electrostatic and hydrogen bridge interactions that took place in transmembrane and cytoplasmic regions of the CD133 protein were excluded since, in accordance with the present invention, such data are not relevant.

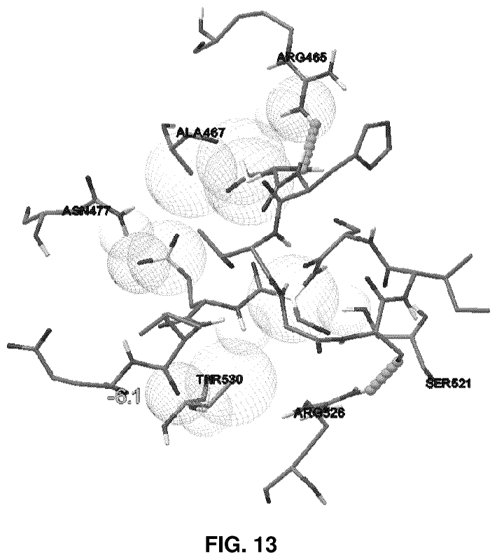

The results of the electrostatic and hydrogen bridge interactions presented between the peptides ABCP-1, ABCP-2, ABCP-3 and ABCP-4 with CD133, are shown in FIGS. 11 to 14; as can be observed, the protein chain of CD133 and some segments of the peptides of the invention are represented in the form of a line that is observed in gray color, where the amino and carboxyl groups of the amino acids are shown in red and blue color, respectively. The blue and red spheres symbolize the electrostatic interactions between the two molecules, the latter being stronger than the former. The hydrogen bridges are shown in small green spheres that form a bridge or bond between the two molecules. For FIG. 13, it is marked with a yellow star is being excluded because it is located in the second transmembrane region of CD133.

Tables 3 to 6 detail the interactions observed between the peptides of the invention and CD133.

Table 3 shows the results of molecular docking between ABCP-1 and CD133. Eight electrostatic interactions of ABCP-1 towards CD133, 16 electrostatic interactions of CD133 towards ABCP-1 and four hydrogen bridge bonds were found. It was possible to represent in a figure all the interactions shown between both molecules (FIG. 11), where such interactions between the amino acids of ABCP-1 and CD133 are shown.

The second and third columns correspond to the interactions of the amino acids of ABCP-1 with the amino acids of CD133. The third and fourth columns show the interaction between the amino acids of CD133 and ABCP-1. The last section of the table shows the hydrogen bonding that occurred between the two molecules and the amino acids that act as electron donors and acceptors to achieve this bonding.

TABLE 3
Molecular interactions between ABCP-1 and CD133.

| | Close interactions between | | Close interactions between | |
|---|---|---|---|---|
| Contact | ABCP-1 | | Amino acids | Amino acids |
| No. | amino acids | CD133 Amino Acids | of CD133 | of ABCP-1 |
| 1 | HIS8 | SER312, ARG399, ASN395 | GLN403 | ASN1 |
| 2 | GLN5 | LEU321, ARG320 | LEU436 | HIS8 |
| 3 | TYR2 | ASP366, ALA408, GLN362 | LEU321 | GLN5 |
| 4 | ASN1 | GLN403, ASP404 | ASP366 | GLU3, TYR2 |
| 5 | ALA10 | HIS310, SER432 | ARG399 | SER7, ASP6, HIS8 |
| 6 | GLU3 | SER324, ASP366, SER328 | ASN395 | HIS8 |
| 7 | ASP6 | ARG399, ASN317 | ASN317 | ASP6 |
| 8 | SER7 | ARG320, ARG399, SER312 | SER324 | GLU3 |
| 9 | | | SER432 | ALA10 |
| 10 | | | ARG320 | SER7, GLN5 |
| 11 | | | GLN362 | TYR2 |
| 12 | | | ASP404 | ASN1 |
| 13 | | | SER328 | GLU3 |
| 14 | | | HIS310 | ALA10 |
| 15 | | | ALA408 | TYR2 |
| 16 | | | SER312 | HIS8, SER7 |

Hydrogen bonding

| | Amino acid donor | Amino acid acceptor |
|---|---|---|
| 1 | ALA10 | HIS310 |
| 2 | HIS310 | ALA10 |
| 3 | SER328 | GLU3 |
| 4 | ARG399 | HIS8 |

Table 4 shows the results of molecular docking between ABCP-2 and CD133. Seven electrostatic interactions of ABCP-2 towards CD133, eleven electrostatic interactions of CD133 towards ABCP-2 and one hydrogen bridge bond were found. All the interactions shown between the amino acids of ABCP-2 and CD133 were also represented in FIG. 12. The second and third columns correspond to the interactions of the amino acids of ABCP-2 with the amino acids of CD133. The third and fourth columns show the interaction between the amino acids of CD133 and ABCP-2. The last section of the table shows the hydrogen bridge bonding that occurred between the two molecules and the amino acids that act as electron donors and acceptors to achieve this bonding.

The observed interactions between ABCP-3 and CD133 are shown in FIG. 13.

TABLE 4
Molecular interactions between ABCP-2 and CD133.

| | Close contact between: | | Close contact between: | |
|---|---|---|---|---|
| Contact | ABCP-2 | Amino acids | Amino acids | Amino acids |
| No. | amino acids | of CD133 | of CD133 | of ABCP-2 |
| 1 | VAL1 | SER543, GLU539 | SER543 | VAL1, TYR2 |
| 2 | TYR2 | SER543 | GLY592 | LYS7 |
| 3 | ASP4 | SER593 | GLU539 | VAL1 |
| 4 | LYS7 | ILE587, TYR634, | SER593 | ASP4 |
| | | GLY592, LEU647, | | |
| | | VAL645 | | |
| 5 | TYR12 | ASN659, ALA656, | TYR634 | LYS7 |
| | | LYS700 | | |
| 6 | ASN8 | VAL645 | LYS700 | TYR12 |
| 7 | LYS6 | ILE587 | ILE587 | LYS7, LYS6 |
| 8 | | | ASN659 | TYR12 |
| 9 | | | ALA656 | TYR12 |
| 10 | | | LEU647 | LYS7 |
| 11 | | | VAL645 | LYS7, ASN8 |

Hydrogen bonding

| | Amino acid | Amino acid |
|---|---|---|
| | donor | acceptor |
| 1 | VAL645 | LYS7 |

Table 5 shows the results of molecular docking between ABCP-3 and CD133. Four electrostatic interactions of ABCP-3 towards CD133, three electrostatic interactions of CD133 towards ABCP-3 and one hydrogen bridge bond were found. The second and third columns correspond to the interactions of the amino acids of ABCP-3 with the amino acids of CD133. The third and fourth columns show the interaction between the amino acids of CD133 and ABCP-3. The last section of the table shows the hydrogen bridge bonding that occurred between the two molecules and the amino acids that act as electron donors and acceptors to achieve this bonding.

TABLE 5
Molecular interactions between ABCP-3 and CD133.

| | Close contact between: | | Close contact between: | |
|---|---|---|---|---|
| Contact | Amino acids | Amino acids | Amino acids | Amino acids |
| [symbol] | of ABCP-3 | of CD133 | of CD133 | of ABCP-3 |
| 1 | GLU9 | THR530 | SER521 | SER8 |
| 2 | SER8 | SER521 | ARG526 | SER5 |
| 3 | SER5 | ARG526 | THR530 | LEU10, GLU9 |
| 4 | LEU10 | THR530 | | |

Hydrogen bonding

| | Amino acid donor | Amino acid acceptor |
|---|---|---|
| 1 | ARG526 | SER5 |

[symbol]

indicates data missing or illegible when filed

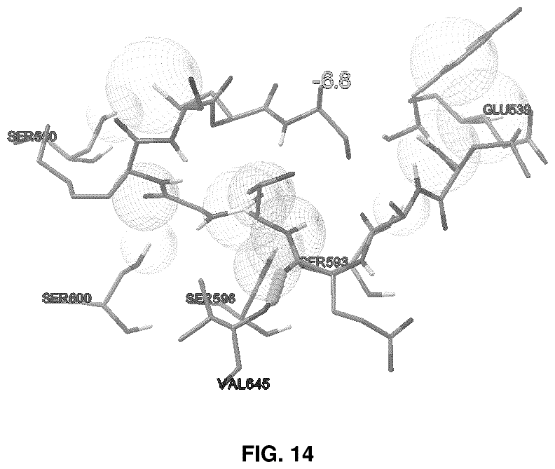

Finally, Table 6 shows the results of molecular docking between ABCP-4 and CD133. Five electrostatic interactions of ABCP-4 towards CD133, six electrostatic interactions of CD133 towards ABCP-4 and one hydrogen bridge bond were found. All observed interactions between ABCP-4 and CD133 are shown in FIG. 14. The second and third columns correspond to the interactions of the amino acids of ABCP-4 with the amino acids of CD133. The third and fourth columns show the interaction between the amino acids of CD133 and ABCP-4. The last section of the table shows the hydrogen bridge bonding that occurred between the two molecules and the amino acids that act as electron donors and acceptors to achieve this bonding.

TABLE 6
Molecular interactions between ABCP-4 and CD133.

| | Close contact between: | | Close contact between: | |
|---|---|---|---|---|
| Contact | Amino acids | Amino acids | Amino acids | Amino acids |
| No. | of ABCP-4 | of CD133 | of CD133 | of ABCP-4 |
| 1 | THR5 | SER596 | SER593 | TYR1 |
| 2 | PRO9 | SER550 | VAL645 | GLN4 |
| 3 | GLY6 | SER600 | GLU539 | TYR1 |
| 4 | TYR1 | SER593, GLU539 | SER596 | THR5 |
| 5 | GLN645 | VAL645 | SER600 | GLY6 |
| | | | SER550 | PRO9 |

Hydrogen bonding

| | Amino acid donor | Amino acid acceptor |
|---|---|---|
| 1 | VAL645 | GLN4 |

### Test of Effectiveness of the Peptides of the Invention on Tumor Cells.

In order to demonstrate the specificity of the peptides of the invention, their effectiveness was tested on different cell lines of neoplastic origin (U87-MG, HEp2, U2-OS) and kidney cells of non-tumor origin (VERO). The corresponding cell line grown in peptide-free medium was used as a test control.

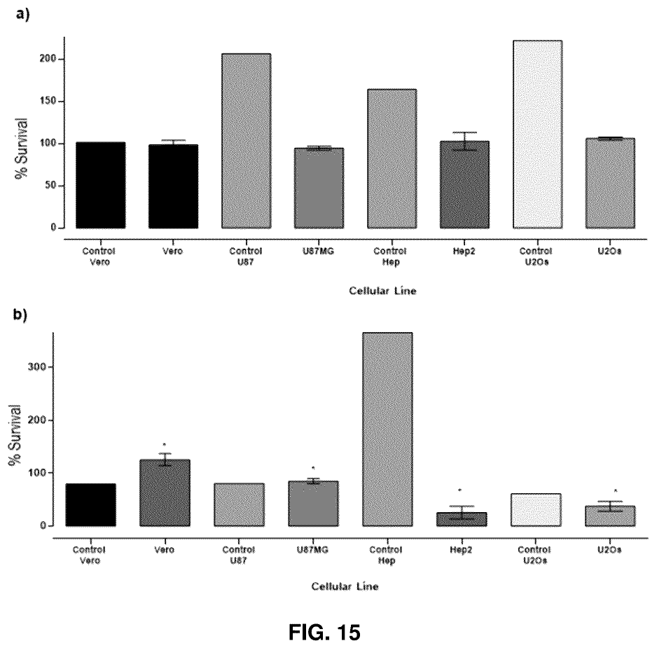

FIG. 15 shows the percentage of survival in different cell lines exposed to ABCP-1 and ABCP-4 peptides with an initial concentration of 40.8 mg for 48 h. Four cell lines (U87-MG, Glioblastoma; HEp2, laryngeal cancer; and U2-OS, Osteosarcoma), were compared with their cell control (cells with growth medium WITHOUT peptide) and against cells of non-neoplastic origin (Vero).

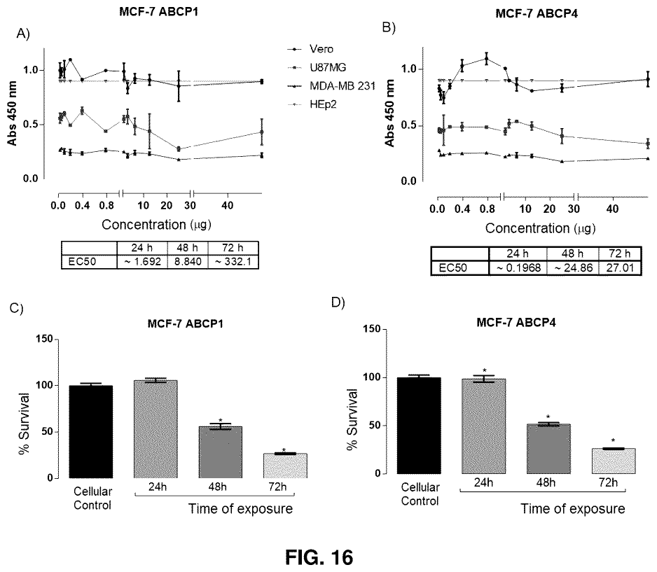

FIG. 16 shows the effect of ABCP1 (A) and ABCP4 (B) peptides on cell viability of the MCF-7 line and its percentage survival. The graphs show the mean±SEM. Figures A and B were analyzed by 3-parameter nonlinear regression, determining the CI₅₀ at each time. Figures C and D were analyzed by a two-factor ANOVA with a post hoc Dunnet's test (*) p<0.05 with respect to the control. Black shows the reading at 24 hours, red at 48 hours and blue at 72 hours, cell control in green.

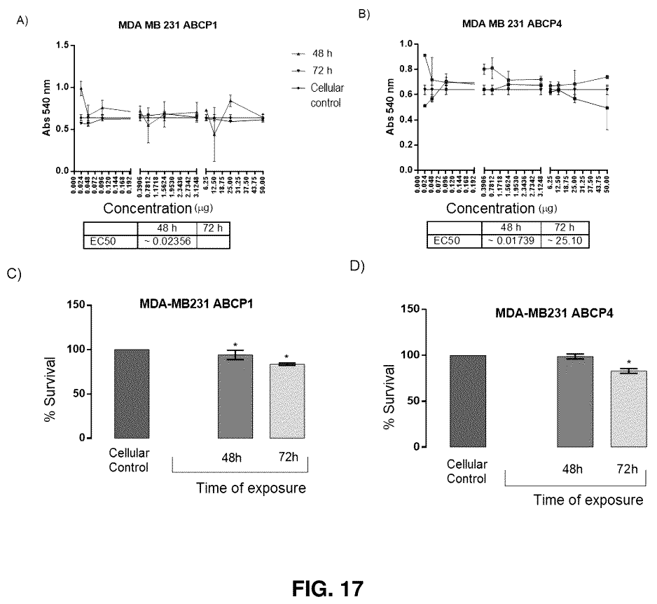

FIG. 17 shows the effect of ABCP1 (A) and ABCP4 (B) peptides on cell viability of the MDA-MB231 line and its percentage survival. The graphs show the mean±SEM. Figures A and B were analyzed by 3-parameter nonlinear regression, determining the CI₅₀ at each time. Figures C and D were analyzed by a two-factor ANOVA with a post hoc Dunnet's test (*) p<0.05 with respect to the control.

The effectiveness of the ABCP-1 and ABCP-4 peptides of the invention had an effect on triple negative breast cancer cells (FIG. 17), as well as on other cancers, as can be seen in FIG. 15(a,b) where ABCP-1 peptide reduces viability in U87-MG, HEP-2 and U2Os cells (glioblastoma, laryngeal cancer and osteosarcoma, respectively), while ABCP-4 peptide significantly reduces viability in HEp2 and U2Os cells. It is also observed that the amount of these peptides is extremely low, which is confirmed by the CI values₅₀ obtained for the peptides ABCP-1 (0.02356 μg to 1.692 μg) and ABCP-4 (0.01739 μg to 0.1968 μg) for MCF-7 and MDA-MB231 breast cancer cells (FIGS. 16 and 17), where even lower concentration of these peptides was required in MDA-MB231 cells (triple negative) to observe their anticancer effect. This result makes it possible to administer these peptides in extremely low amounts, which implies the elimination of possible side effects that could be generated in the patient to be treated by the possible administration of larger amounts of active principles.

Likewise and as seen in Tables 7 and 8, the peptides of the invention significantly reduce the survival percentage of various types of cancer cells (by at least 37%, preferably from 37.14% to 93.04%), which allows them to be used in a versatile manner for the treatment of cancers such as, for example, glioblastoma (U87-MG), laryngeal cancer (HEP-2), osteosarcoma (U2Os), breast cancer (MCF-7) and triple-negative breast cancer (MDA-MB231).

It is worth considering that CD133 over expression is downregulated in various solid tumors; however, despite numerous studies, the role of this surface antigen in tumorogenesis and tumor progression is largely unknown (Liou G. Y.), for such reason the biological effect of the ABCP-1 and ABCP-4 peptides of the present invention is not anticipatable from the prior art.

The effect on the viability of the MDA-MB231 cell line (triple negative breast cancer) was not drastic but was statistically significant (FIG. 17), possibly due to the deregulation of the WNT oncogene present in this cell lineage, which is known to activate the expression of CD133 and thus reduce the effect of the peptides designed against this molecule; however, this behavior should be tested in other cell lineages derived from the MDA-MB line, since each of these has different oncogenes.

TABLE 7
Percentages of cancer cell survival with the
most common peptides of the invention.

| | | | % reduction in survival* | |
|---|---|---|---|---|
| | | | % reduction in survival* | |
| | | | % reduction in survival* | |
| | | | % reduction in survival* | |
| | | | % reduction in survival* | |
| | Survival %*. | | % reduction in survival | |
| Cellular type | ABCP-1 | ABCP-4 | ABCP-1 | ABCP-4 |
| Vero Control | 100 | 100 | — | — |
| Vero | 96.45 | 100 | 3.54 | 0 |
| U87-MG Control | 100 | 100 | — | — |
| U87-MG | 45.78 | 100 | 54.22 | 0 |
| HEP-2 Control | 100 | 100 | — | — |
| HEP-2 | 62.60 | 6.96 | 37.41 | 93.04 |
| U2Os Control | 100 | 100 | — | — |
| U2Os | 47.71 | 60.77 | 52.30 | 39.24 |

*Regarding Control

TABLE 8
Percentage survival rates of breast cancer cells at different
exposure times with the peptides of the invention.

| | | % reduction in survival* | |
|---|---|---|---|
| | | % reduction in survival* | |
| | | % reduction in survival* | |
| | | % reduction in survival* | |
| | | % reduction in survival* | |
| Survival %*. | | % reduction in survival | |
| ABCP-1 | ABCP-4 | ABCP-1 | ABCP-4 |

MCF-7

| | | | | |
|---|---|---|---|---|
| Cellular witness | 100 | 100 | — | — |
| 24 hrs | 100 | 98.64 | 0 | 1.36 |
| 48 hrs | 56.04 | 51.59 | 43.96 | 48.41 |
| 72 hrs | 26.79 | 26.26 | 73.21 | 73.74 |

MDA-MB231

| | | | | |
|---|---|---|---|---|
| Cellular witness | 100 | 100 | — | — |
| 48 hrs | 93.98 | 93.98 | 6.02 | 6.02 |
| 72 hrs | 83.60 | 83.60 | 16.4 | 16.4 |

*Regarding Control

In accordance with the present invention, we found that indeed the effect of the designed peptides of the invention on cells from non-triple negative breast cancer (MCF-7 breast tissue adenocarcinoma cells), such as ABCP-1 and ABCP-4 peptides, are able to reduce the viability of this neoplasm, an effect dependent on time and dose of exposure (FIG. 16).

The following examples are included below for the sole purpose of illustrating the invention, without implying limitations on its scope.

### Effect of ABCP-1 in Breast Cancer Murine Model:

Preclinical trial was followed as described in example 5, tumor induction was performed by inoculating 4T1 cells (20,000 4T1 cells) in mammary gland of 8-week-old female Balb/c mice. The tumor growth and metastatic spread of 4T1 cells in BALB/c mice very closely mimic human breast cancer. This tumor-derived cell line is an animal model for stage IV human breast cancer.

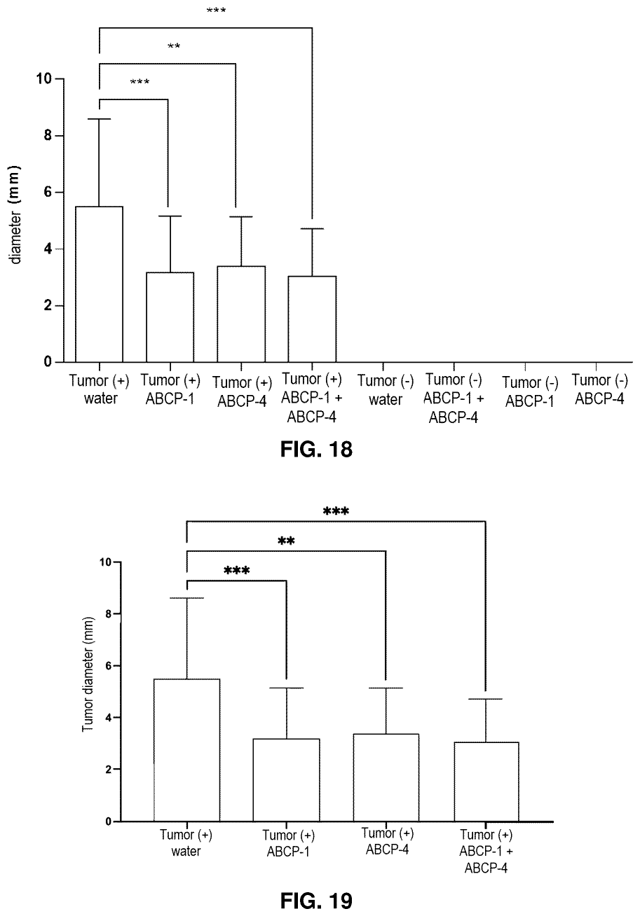

In FIG. 18 it is shown that none of the study groups showed tumor development when inoculated only with WATER (absence of tumor cells) followed with the administration of ABCP-1, ABCP-4, or the mixture of both; that is, the peptides by themselves are not able to generate hyperplasia or any neoplasia (non-tumorigenic) during 14 days of follow-up.

In FIG. 19 it is shown the reduction in tumor diameter in mice treated with the peptides ABCP-1, ABCP-4, or the mixture of both, compared to the group of tumors treated with water, was statistically significant (p<0.05) according to one-way ANOVA.

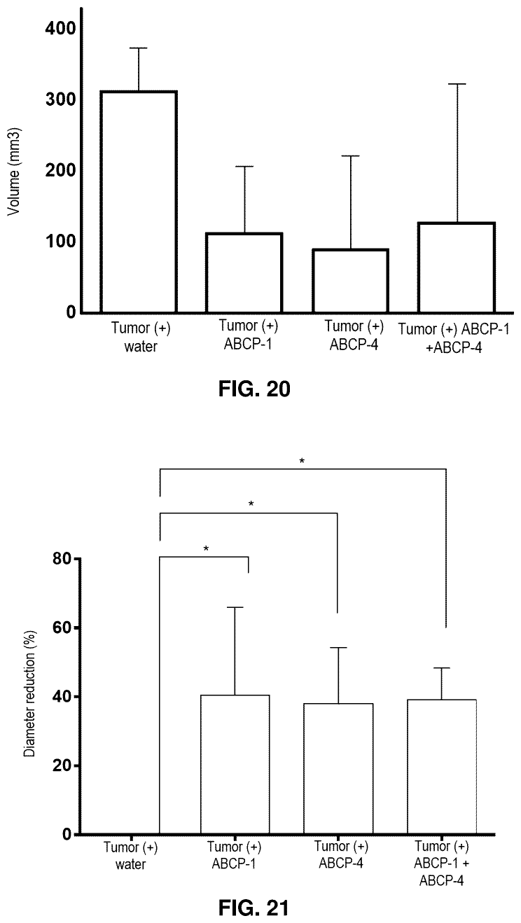

FIG. 20 shows the reduction in tumor volume in mice treated with the peptides ABCP-1, ABCP-4, or the mixture of both, compared to the group treated with water, may not be significant because one of the mice treated with the ABCP-4 peptide and another of the individuals treated with the mixture of both peptides (ABCP-1 and ABCP-4) developed minimal tumor growth without quantifiable increase using the applied methodology (digital caliper).

FIG. 21 shows the percentage reduction in tumor diameter in mice treated with the peptides ABCP-1, ABCP-4, or the mixture of both, compared to the group of tumors treated with water, is statistically significant (p<0.05) according to one-way ANOVA. Percentage reduction with the ABCP-1 peptide of up to 64% with an average of 40.57%, Percentage reduction with the ABCP-4 peptide of up to 54% with an average of 38.03%, Percentage reduction with the mixture of ABCP-1 and ABCP-4 peptides of up to 44% with an average of 39.22%.

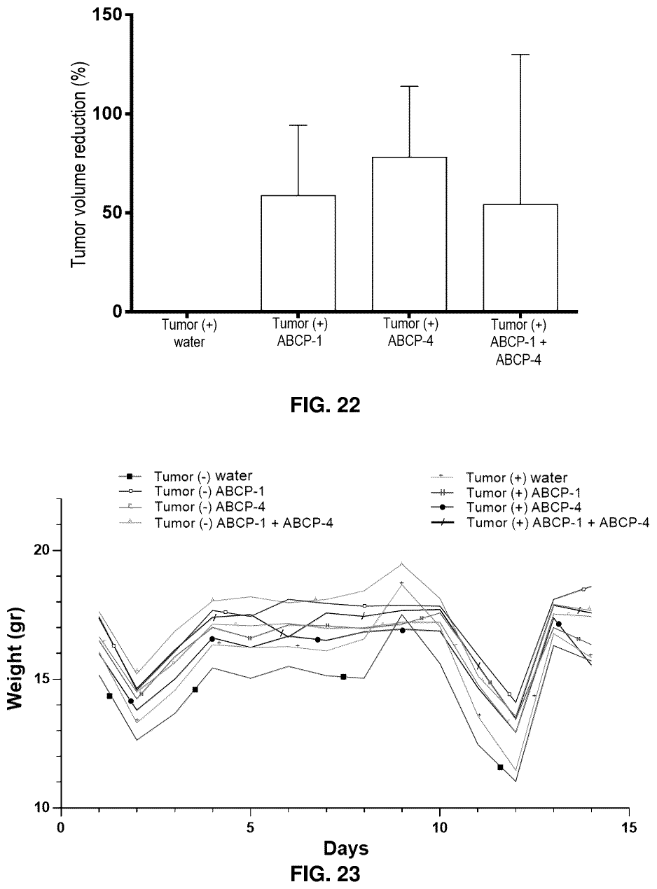

FIG. 22 shows the percentage of reduction in tumor volume in those mice treated with the peptides ABCP-1, ABCP-4, or a mixture of both, compared to the group treated with water, may not be significant because one of the mice treated with the ABCP-4 peptide and another of the mice treated with the mixture of both peptides (ABCP-1 and ABCP-4) developed minimal tumor growth without a quantifiable increase using the applied methodology (digital caliper).

### Example 1. Previous Cell Cultures to Evaluate Peptide Treatments

Cell subculture: MDA-MB-231 (breast cancer), MCF-7 (breast cancer), U87-MG (glioblastoma), HEp2 (laryngeal carcinoma) and U2-OS (osteosarcoma) cell lines were seeded separately in 75 cm bottles², using DMEM-F12 medium supplemented with 10%, 1% non-essential amino acids, 1% L-glutamine, 1% penicillin-streptomycin and 1% amphotericin B, incubated at 37° C. with an atmosphere containing 5% CO₂ until approximately 90% confluence was achieved.

### Example 2. Determination of the CI₅₀% on Different Cell Lines

The cells of each cell line already cultured were adjusted to 5×10⁴ cells/well in 96-well plates, using one row for each cell line up to well 10, where wells 11 and 12 of each row can be the cell controls and the reagent blank, respectively; after plating they were incubated at 37° C. for 24 hours; at the end of this time the peptides of the invention were added, starting at a concentration of 200 mg/mL, making dilutions 1:2 starting from well 1 to well 10 and bringing to a final volume of 200 μL, a cell control with medium without peptide and a reagent blank without cells.

The cell lines were exposed to the peptides of the invention (treatments) for different times (24, 48 and 72 hours). At the end of each time, 50 μL of MTT were added, incubated for 4 hours at 37° C., 5% CO₂, and then 100 μL of SDS-HCl were added, incubated at room temperature overnight, and finally readings were performed in an ELISA reader at 450 nm and 500 nm.

The calculation of the ICcc50% was performed as follows: once the averages of the absorbance readings were obtained for each case, the average of the reagent blank readings was subtracted, and the following formula was used:

$$ICcc 50 \% = ( A - B / B ) \times 100 \qquad 1$$

where A=Average of the peptides at a certain concentration (separately, ABCP-1 and ABCP-4 peptide, perform the corresponding calculations for each peptide in its respective assay), B=Average of the cell control. The CI_{CC} 50% was obtained by linear regression.

### Example 3: Post-Treatment Cell Viability

MDA-MB 231 and MCF-7 cell lines were seeded (5×10^{4 c} cells/well) for subsequent incubation at 37° C. in 5% CO₂ for 24 hours. After the incubation time had elapsed, the peptides of the invention were added. Starting from an initial concentration of 40.8 μg/200 μL and performing serial dilutions with a 1:2 factor using maintenance medium without serum as diluent until reaching a concentration of 0.08 μg/200 μL, the peptides of the invention (ABCP-1 and ABCP-4) were inoculated, using separate plates. Once the peptide was inoculated, the plates were incubated for 24, 48 and 72 hours at 37° C. with 5% CO₂. After each incubation time had elapsed, MTT was plated as described above and the corresponding CI₅₀ was calculated for each cell line.

### Example 4: Percentage of Cell Death

Considering the absorbances of the cell targets (only with the maintenance medium, without peptide) as 100% viability, the calculation of post-treatment survival and thus the percentage of death was performed using the following equations:

$$survival \% = ( Abs problem / Abs control ) * 100 \qquad 2$$

$$\% of death = 100 - \% survival \qquad 3$$

### Example 5: Treatment Scheme in 9-Week-Old Balb/c Mice

Prior to the start of treatment, tumor induction was performed by inoculating 4T1 cells to each mice group as follows: 8-week-old female Balb/c mice were inoculated with 20,000 4T1 cells previously resuspended in 20 μL of sterile PBS pH 7.4, the inoculation was subdermal performed in the second left mammary pad using an insulin syringe. This breast cancer preclinical model has 100% efficiency, generating in mice a tumor with central necrosis and ulceration, which at 4 weeks of development resembles stage IV human breast cancer with local and distal invasion, with metastases mainly to the lung and bone, and occasionally to the brain.

Treatment scheme in 9-week-old Balb/c mice is shown in table 9. Prior to the start of treatment, tumor induction was performed by inoculating 4T1 cells to each mice group as follows: 8-week-old female Balb/c mice were inoculated with 20,000 4T1 cells previously resuspended in 20 μL of sterile PBS pH 7.4, the inoculation was performed subdermally in the second left mammary pad using an insulin syringe. This breast cancer preclinical model has 100% efficiency, generating in mice a tumor with central necrosis and ulceration, which at 4 weeks of development resembles stage IV human breast cancer with local and distal invasion, with metastases mainly to the lung and bone, and occasionally to the brain.

TABLE 9
Treatment scheme in 9-week-old Balb/c mice.

| | | | Peptide |
|---|---|---|---|
| Study Mice | Balb/c Mice | Administered | concentration |
| Group | tumor (+)/(−) | drug | (□g/mL) |
| 1 (n = 3) | Tumor (+) | ABCP-1 | 13.95 |
| 2 (n = 3) | Tumor (+) | ABCP-4 | 5.86 |
| 3 (n = 3) | Tumor (+) | ABCP-1 + ABCP-4 | 13.95 + 5.86 |
| 4 (n = 3) | Tumor (+) | Inyectable water | 0 |
| 5 (n = 3) | without tumor | ABCP-1 | 13.95 |
| 6 (n = 3) | without tumor | ABCP-4 | 5.86 |
| 7 (n = 3) | without tumor | ABCP-1 + ABCP-4 | 13.95 + 5.86 |
| 8 (n = 3) | without tumor | Inyectable water | 0 |

All groups received the 1^{st} peptide administration at day 7 post celular inoculation.
All groups received the 2^{nd} peptide administration at day 14 post celular inoculation.

### Used Concentrations of the Peptides

Peptide ABCP-1 (13.95 μg/mL)
Peptide ABCP-4 (5.86 μg/mL)
Peptide ABCP-1+ABCP-4 (13.95 μg/mL+5.86 μg/mL)
Serum quantity: 50 μL recovered by mouse.
Inoculated cell line: 4T1
Test duration: 21 days.

As shown in FIG. 23, none of the study groups showed weight loss, meaning that the administration of ABCP-1 and ABCP-4 does not seem to have side effects such as anorexia, vomiting, or dehydration.

### REFERENCES

ACS. 2022. American Cancer Society. https://www.cancer.org/cancer/breast-cancer/about/what-is-breast-cancer.html.
ACS. 2022. American Cancer Society. https://www.cancer.org/cancer/breast-cancer/about/types-of-breast-cancer/triple-negative.html.
Barzegar-Behrooz, A., et. al. 2019. Journal of Drug Targeting, 27 (3), 257-269. Breast Cancer. 2022. Types of Breast Cancer. https://www.breastcancer.org/types/male-breast-cancer.
Clarck, J. W. et. al. 2012. Cancer Cell Biology. In F. H. Kasper, Harrison's: Principles on Internal Medicine (19th ed., p. 483). McGraw Hill Education.
Cleveland Clinic. 2022. Cleveland Clinic. https://my.clevelandclinic.org/health/diseases/3986-breast-cancer
Foulkes, W. et. al. 2010. New England Journal Medicine, 363 (20), 1938-1948.
Glumac, P. M., et. al. 2018. Clinical and translational Medicine, 7 (18), 1-14.
INEGI. 2021. Press Release No. 571/21. https://www.inegi.org.mx/contenidos/saladeprensa/aproposito/2021/EAP_LUCHACANCER2021.pdf
INEGI. 2022. Press Release No. 143/22. https://www.inegi.org.mx/contenidos/saladeprensa/aproposito/2022/EAP_Mujer22.pdf
INEGI. 2022. Press Release No. 74/22. https://www.inegi.org.mx/contenidos/saladeprensa/aproposito/2022/EAP_CANCER22.pdf
Jae-Woo, J., et. al. 2017. Life Sciences, 184, 25-29.
Kanwal, R., et. al. 2018. Cancer Letters, 430, 25-33.
Liou, G.-Y. 2019. Int J Biochem Cell Biol, 106, 1-4.
Marqus, S., et. al. 2017. Journal of Biomedical Science, 24 (21), 1-15.
PAHO. 2020. Pan American Health Organization. http://WHO.int
Reyes Bacardi, K., et. al. 2013. Rev Cubana Genet Comunit, 7 (2), 4-11.
Weigelt, B., et. al. 2010. Molecular Oncology, 4, 192-208.
WHO. 2018. World Health Organization. https://www.who.int/news-room/fact-sheets/detail/cancer.
WHO. 2020. World Health Organization. https://www.who.int/cancer/about/facts/en/

1. A peptide with anticancer and CD133 protein antagonist activity, characterized in that it comprises a peptide from the group comprising ABCP-1 peptide with sequence SEQ. ID. NO. 1, ABCP-2 peptide with sequence SEQ. ID. NO. 2, ABCP-3 peptide with sequence SEQ. ID. NO. 3, and ABCP-4 peptide with sequence SEQ. ID. NO. 4.

2. The peptide according to claim 1, characterized in that it is the peptide ABCP-1 with sequence SEQ. ID. NO. 1.

3. The peptide according to claim 1, characterized in that it is the peptide ABCP-4 with sequence SEQ. ID. NO. 4.

4. A pharmaceutical composition characterized in that it comprises the peptide of claim 1 and a pharmaceutically compatible vehicle.

5. A nucleic acid sequence, characterized in that it encodes for the peptide according to claim 1.

6. The peptide according to claim 1, for use in the treatment of cancer.

7. The peptide for use according to claim 6, wherein the cancer is selected from the group comprising glioblastoma, laryngeal cancer, osteosarcoma, breast cancer and triple negative breast cancer.

8. The pharmaceutical composition according to claim 4, for use in the treatment of cancer.

9. The pharmaceutical composition for use according to claim 8, wherein the cancer is selected from the group comprising glioblastoma, laryngeal cancer, osteosarcoma, breast cancer and triple negative breast cancer.

10. The use of the peptide according to claim 1, for the manufacture of a medicament for the treatment of cancer.

11. The use of the peptide according to claim 10, wherein the cancer is selected from the group comprising glioblastoma, laryngeal cancer, osteosarcoma, breast cancer and triple negative breast cancer.

12. A method for treating cancer, wherein that it comprises the step of administering the peptide according to claim 1, to a patient suffering from cancer.

13. The method according to claim 12, wherein that the cancer is selected from the group comprising glioblastoma, laryngeal cancer, osteosarcoma, breast cancer and triple negative breast cancer.

14. A method for treating cancer, characterized in that it comprises the step of administering the composition according to claim 4, to a patient suffering from cancer.

15. The method according to claim 14, characterized in that the cancer is selected from the group comprising glioblastoma, laryngeal cancer, osteosarcoma, breast cancer and triple negative breast cancer.
